SEQUENCE LISTING (51 sequences)

SEQ ID NO: 1 - protein, 501 aa, Artificial Sequence
  Synthesized
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 2 - DNA, 1506 bp, Artificial Sequence
  Synthesized
atggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtcatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtga

SEQ ID NO: 3 - protein, 21 aa, Artificial Sequence
  Synthesized
MAQALPWLLLWMGAGVLPAHG

SEQ ID NO: 4 - protein, 34 aa, Artificial Sequence
  Synthesized
TQHGIRLPLRSGLGGAPLGLRLPRETDEEPEEPG

SEQ ID NO: 5 - protein, 22 aa, Artificial Sequence
  Synthesized
GTDLVSIPHGPNVTVRANIAAI

SEQ ID NO: 6 - protein, 20 aa, Artificial Sequence
  Synthesized
DLVSIPHGPNVTVRANIAAI

SEQ ID NO: 7 - protein, 18 aa, Artificial Sequence
  Synthesized
DLVSIPHGPNVTVRANIA

SEQ ID NO: 8 - protein, 16 aa, Artificial Sequence
  Synthesized
VSIPHGPNVTVRANIA

SEQ ID NO: 9 - protein, 14 aa, Artificial Sequence
  Synthesized
IPHGPNVTVRANIA

SEQ ID NO: 10 - protein, 12 aa, Artificial Sequence
  Synthesized
IPHGPNVTVRAN

SEQ ID NO: 11 - protein, 10 aa, Artificial Sequence
  Synthesized
HGPNVTVRAN

SEQ ID NO: 12 - protein, 243 aa, Artificial Sequence
  Synthesized
SNSMFRGLRDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPSGLFKVSKLISDLDSTAVYAQ

SEQ ID NO: 13 - protein, 235 aa, Artificial Sequence
  Synthesized
RDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPSGLFKVSKLISDLDSTAVYAQ

SEQ ID NO: 14 - protein, 215 aa, Artificial Sequence
  Synthesized
RDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPS

SEQ ID NO: 15 - protein, 214 aa, Artificial Sequence
  Synthesized
RDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQP

SEQ ID NO: 16 - protein, 211 aa, Artificial Sequence
  Synthesized
RDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQA

SEQ ID NO: 17 - protein, 207 aa, Artificial Sequence
  Synthesized
RDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSL

SEQ ID NO: 18 - protein, 214 aa, Artificial Sequence
  Synthesized
RDYNPISNNICHLTNVSDGASNSLYGVGFGPLILTNRHLFERNNGELVIKSRHGEFVIKNTTQLHLLPIPDRNLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQP

SEQ ID NO: 19 - protein, 214 aa, Artificial Sequence
  Synthesized
RDYNPISNNICHLTQVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQP

SEQ ID NO: 20 - protein, 214 aa, Artificial Sequence
  Synthesized
RDYNPISNNICHLTNVSDGASNSLYGVGFGPLILTNRHLFERNNGELVIKSRHGEFVIKQTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQP

SEQ ID NO: 21 - protein, 214 aa, Artificial Sequence
  Synthesized
RDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYQTSAISWGSLNIQASQP

SEQ ID NO: 22 - protein, 9 aa, Artificial Sequence
  Synthesized
QTDESTLMT

SEQ ID NO: 23 - protein, 8 aa, Artificial Sequence
  Synthesized
TDESTLMT

SEQ ID NO: 24 - protein, 8 aa, Artificial Sequence
  Synthesized
QTDESTLM

SEQ ID NO: 25 - protein, 7 aa, Artificial Sequence
  Synthesized
DESTLMT

SEQ ID NO: 26 - protein, 7 aa, Artificial Sequence
  Synthesized
QTDESTL

SEQ ID NO: 27 - protein, 6 aa, Artificial Sequence
  Synthesized
ESTLMT

SEQ ID NO: 28 - protein, 6 aa, Artificial Sequence
  Synthesized
QTDEST

SEQ ID NO: 29 - protein, 21 aa, Artificial Sequence
  Synthesized
IAYVMAAICALFMLPLCLMVC

SEQ ID NO: 30 - protein, 23 aa, Artificial Sequence
  Synthesized
QWRCLRCLRQQHDDFADDISLLK

SEQ ID NO: 31 - protein, 335 aa, Artificial Sequence
  Synthesized
MAQALPWLLLWMGAGVLPAHGTQHGIRLPLRSGLGGAPLGLRLPRETDEEPEEPGRDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPNIPQTDESTLMTIAYVMAAICALFMLPLCLMVCQWRCLRCLRQQHDDFADDISLLKEQKLISEEDL

SEQ ID NO: 32 - protein, 335 aa, Artificial Sequence
  Synthesized
MAQALPWLLLWMGAGVLPAHGTQHGIRLPLRSGLGGAPLGLRLPRETDEEPEEPGRDYNPISNNICHLTQVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPNIPQTDESTLMTIAYVMAAICALFMLPLCLMVCQWRCLRCLRQQHDDFADDISLLKEQKLISEEDL

SEQ ID NO: 33 - protein, 335 aa, Artificial Sequence
  Synthesized
MAQALPWLLLWMGAGVLPAHGTQHGIRLPLRSGLGGAPLGLRLPRETDEEPEEPGRDYNPISNNICHLTNVSDGASNSLYGVGFGPLILTNRHLFERNNGELVIKSRHGEFVIKQTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPNIPQTDESTLMTIAYVMAAICALFMLPLCLMVCQWRCLRCLRQQHDDFADDISLLKEQKLISEEDL

SEQ ID NO: 34 - protein, 335 aa, Artificial Sequence
  Synthesized
MAQALPWLLLWMGAGVLPAHGTQHGIRLPLRSGLGGAPLGLRLPRETDEEPEEPGRDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYQTSAISWGSLNIQASQPNIPQTDESTLMTIAYVMAAICALFMLPLCLMVCQWRCLRCLRQQHDDFADDISLLKEQKLISEEDL

SEQ ID NO: 35 - protein, 335 aa, Artificial Sequence
  Synthesized
MAQALPWLLLWMGAGVLPAHGTQHGIRLPLRSGLGGAPLGLRLPRETDEEPEEPGRDYNPISNNICHLTNVSDGASNSLYGVGFGPLILTNRHLFERNNGELVIKSRHGEFVIKNTTQLHLLPIPDRNLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPNIPQTDESTLMTIAYVMAAICALFMLPLCLMVCQWRCLRCLRQQHDDFADDISLLKEQKLISEEDL

SEQ ID NO: 36 - protein, 315 aa, Artificial Sequence
  Synthesized
MAQALPWLLLWMGAGVLPAHGTQHGIRLPLRSGLGGAPLGLRLPRETDEEPEEPGRDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPNIPQTDESTLMTIAYVMAAICALFMLPLCLMVCQWREQKLISEEDL

SEQ ID NO: 37 - protein, 280 aa, Artificial Sequence
  Synthesized
MAQALPWLLLWMGAGVLPAHGTQHGIRLPLRSGLGGAPLGLRLPRETDEEPEEPGRDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPNEQKLISEEDL

SEQ ID NO: 38 - protein, 303 aa, Artificial Sequence
  Synthesized
MAQALPWLLLWMGAGVLPAHGTQHGIRLPLRSGLGGAPLGLRLPRETDEEPEEPGRDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPEQKLISEEDLPDHSAATKPSLFLFLVSLLHIFFK

SEQ ID NO: 39 - protein, 306 aa, Artificial Sequence
  Synthesized
MAQALPWLLLWMGAGVLPAHGTQHGIRDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPNIPQTDESTLMTIAYVMAAICALFMLPLCLMVCQWRCLRCLRQQHDDFADDISLLKEQKLISEEDL

SEQ ID NO: 40 - protein, 303 aa, Artificial Sequence
  Synthesized
METDTLLLWVLLLWVPGSTGDYPYDVPDYARDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPAVGQDTQEVIVVPHSLPFKVVVISAILALVVLTIISLIILIMLWQKKPREQKLISEEDL

SEQ ID NO: 41 - protein, 322 aa, Artificial Sequence
  Synthesized
MTSRLSDPNSSARSDMSVPLYPTASPVSVEAYYSESEDEAANDFLVRMGRQQSVLRRRRRRTRCVGMVIACLLVAVLSGGFGALLMWLLRSNSMFRGLRDYNPISNNICHLTNVSDGASNSLYGVGFGPLILTNRHLFERNNGELVIKSRHGEFVIKNTTQLHLLPIPDRDLLLIRLPKDIPPFPQKLGFRQPEKGERICMVGSNFQTKSITSVVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPEQKLISEEDL

SEQ ID NO: 42 - DNA, 1008 bp, Artificial Sequence
  Synthesized
atggctcaggctctcccctggcttttgctgtggatgggcgcgggagttttgcctgctcatggtactcagcacggtattcgccttccccttcggagtgggttgggcggcgcacctctgggactgcgcttgcctagggagacagacgaggagcctgaagaaccgggtcgcgattataatccgattagtaataatatatgtcatctcacgaacgtatccgacggtgcaagcaacagcctgtacggagtcgggtttgggccgcttattttgacaaaccgacatctctttgagagaaataatggggagcttgtcatcaaaagtcggcacggagaatttgtaataaagaataccacacaactccatcttctcccgattccggatcgcgatcttctcctgatcaggttgccgaaggatatccctccattcccgcagaaacttggattcaggcaaccggaaaagggagaacgcatctgcatggtcgggagcaatttccaaactaagtctatcacaagcgtggtgtctgaaactagtacgataatgccagtagagaatagccagttttggaaacattggatcagcacgaaagacggacaatgcggatctccgatggtgtcaactaaagatggtaaaatactcggcctgcattcacttgccaacttccaaaactcaattaactatttcgccgcattccctgacgactttgccgaaaagtaccttcacactatcgaggcacatgaatgggtaaagcactggaagtataacactagcgcgatctcatgggggtcactgaatattcaagcgtctcagccaaatatacctcaaacggacgagagcactcttatgaccatagcttatgtgatggctgcgatctgtgcgctcttcatgcttcccttgtgcctcatggtctgtcaatggcgatgtctgaggtgccttcgacaacaacatgatgattttgcggacgatatttctcttttgaaagagcagaaacttatttcagaagaggacctctga

SEQ ID NO: 43 - protein, 244 aa, Artificial Sequence
  Synthesized
AKSLMRGLRDFNPIAQTVCRLKVSVEYGTSEMYGFGFGAYIIANHHLFRSYNGSMEVRSMHGTFRVKNLHSLSVLPIKGRDIILIKMPKDFPVFPQKLHFRAPTQNERVCLVGTNFQEKYASSIITETSTTYNIPGSTFWKHWIETDNGHCGLPVVSTTDGCLVGIHSLANNKHTTNYYSAFDEDFESKYLRTNEHNEWVKSWIYNPDTVLWGPLKLKDSTPKGLFKTTKLVQDLIDHDVVVEQ

SEQ ID NO: 44 - protein, 210 aa, Artificial Sequence
  Synthesized
RDFNPVAQTVCRLKVSVEFGTSELYGIGFGAYIIANHHLFKSYNGSMEVRSMHGTFRVKNLHSLSVLPIKGRDIILIKMPKDFPVFPQKLRFRAPKQNERICLVGTNFQEKYASSIITETSTTYNVPGSTFWKHWIETDDGHCGLPVVSTADGCLVGIHSLANNVQTTNYYSAFDEDFESKYLRNNEHNEWIKSWVYNPDTVLWGPLKLK

SEQ ID NO: 45 - protein, 244 aa, Artificial Sequence
  Synthesized
SKSLMKGLRDYNPIAQTICKLKVTSEFGTSEMYGIGFGAYIISNHHLFKSFNGTLEVRSHHGTFKVNNMMALQVKPIAGRDIIIIKMPKDFPVFPQKLHFRSPKNNERMCIVGTNFQEKSASSTITETSATYMVPRSSFWKHWIATDDGHCGLPVVSTIDGKIIGIHSLANNANSENYYAAFDDDFEEKFLRTSEHTDWVKNWRYNPDTVVWGPLKLTESTPKGLFKPTKILEDLFEYNSVREQ

SEQ ID NO: 46 - protein, 210 aa, Artificial Sequence
  Synthesized
RDYNPIAQTICKLKVTSEFGTSEMYGIGFGAYIISNHHLFKSFNGTLEVRSHHGTFKVNNMMALQVKPIAGRDIIIIKMPKDFPVFPQKLHFRSPKNNERMCIVGTNFQEKSASSTITETSATYMVPRSSFWKHWIATDDGHCGLPVVSTIDGKIIGIHSLANNANSENYYAAFDDDFEEKFLRTSEHTDWVKNWRYNPDTVVWGPLKLT

SEQ ID NO: 47 - protein, 246 aa, Artificial Sequence
  Synthesized
AKSLLKGLRDYNATAQTVCLLTVETEVGQTTTHGIGFGSLLVANHHLFKSFNGTLTVRSHHGTFKVPNLMQLQVQPLKGRDLIVIKMPKDFPVFPQRLHFRAPTQQDRVVLVGSNFQEKYISTTLSETSSTFPVQRSSFWKHWISTDDGNCGLPLVSTADGYILGLHSLANNRNSENYFAAFDDEFEDRYLRTEEHAQWTRNWKYNPDNVLWGSLKLTKSTPDGMFKTTKMIEDLAAFKIDEVHEQ

SEQ ID NO: 48 - protein, 210 aa, Artificial Sequence
  Synthesized
RDYNATAQTVCLLTVETEVGQTTTHGIGFGSLLVANHHLFKSFNGTLTVRSHHGTFKVPNLMQLQVQPLKGRDLIVIKMPKDFPVFPQRLHFRAPTQQDRVVLVGSNFQEKYISTTLSETSSTFPVQRSSFWKHWISTDDGNCGLPLVSTADGYILGLHSLANNRNSENYFAAFDDEFEDRYLRTEEHAQWTRNWKYNPDNVLWGSLKLT

SEQ ID NO: 49 - protein, 336 aa, Artificial Sequence
  Synthesized
MAQALPWLLLWMGAGVLPAHGTQHGIRLPLRSGLGGAPLGLRLPRETDEEPEEPGLQRDFNPVAQTVCRLKVSVEFGTSELYGIGFGAYIIANHHLFKSYNGSMEVRSMHGTFRVKNLHSLSVLPIKGRDIILIKMPKDFPVFPQKLRFRAPKQNERICLVGTNFQEKYASSIITETSTTYNVPGSTFWKHWIETDDGHCGLPVVSTADGCLVGIHSLANNVQTTNYYSAFDEDFESKYLRNNEHNEWIKSWVYNPDTVLWGPLKLKAAANIPQTDESTLMTIAYVMAAICALFMLPLCLMVCQWRCLRCLRQQHDDFADDISLLKEQKLISEEDL

SEQ ID NO: 50 - protein, 336 aa, Artificial Sequence
  Synthesized
MAQALPWLLLWMGAGVLPAHGTQHGIRLPLRSGLGGAPLGLRLPRETDEEPEEPGLQRDYNPIAQTICKLKVTSEFGTSEMYGIGFGAYIISNHHLFKSFNGTLEVRSHHGTFKVNNMMALQVKPIAGRDIIIIKMPKDFPVFPQKLHFRSPKNNERMCIVGTNFQEKSASSTITETSATYMVPRSSFWKHWIATDDGHCGLPVVSTIDGKIIGIHSLANNANSENYYAAFDDDFEEKFLRTSEHTDWVKNWRYNPDTVVWGPLKLTAAANIPQTDESTLMTIAYVMAAICALFMLPLCLMVCQWRCLRCLRQQHDDFADDISLLKEQKLISEEDL

SEQ ID NO: 51 - protein, 336 aa, Artificial Sequence
  Synthesized
MAQALPWLLLWMGAGVLPAHGTQHGIRLPLRSGLGGAPLGLRLPRETDEEPEEPGLQRDYNATAQTVCLLTVETEVGQTTTHGIGFGSLLVANHHLFKSFNGTLTVRSHHGTFKVPNLMQLQVQPLKGRDLIVIKMPKDFPVFPQRLHFRAPTQQDRVVLVGSNFQEKYISTTLSETSSTFPVQRSSFWKHWISTDDGNCGLPLVSTADGYILGLHSLANNRNSENYFAAFDDEFEDRYLRTEEHAQWTRNWKYNPDNVLWGSLKLTAAANIPQTDESTLMTIAYVMAAICALFMLPLCLMVCQWRCLRCLRQQHDDFADDISLLKEQKLISEEDL

(19) United States
(12) Patent Application Publication
(10) Pub. No.: US 2022/0389400 A1
(43) Pub. Date: Dec. 8, 2022

(54) SYNTHETIC ALPHA-SECRETASE AND USE THEREOF

(71) Applicants: GWANGJU INSTITUTE OF SCIENCE AND TECHNOLOGY, Gwangju (KR); INDUSTRY FOUNDATION OF CHONNAM NATIONAL UNIVERSITY, Gwangju (KR)

(72) Inventors: Woo Jin PARK, Gwangju (KR); Sung Bin KIM, Gwangju (KR); Sung Yun KIM, Gwangju (KR); Won Seok CHOI, Gwangju (KR)

(21) Appl. No.: 17/775,101

(22) PCT Filed: Nov. 6, 2020
(86) PCT No.: PCT/KR2020/015531

§ 371 Date: May 6, 2022

(30) Foreign Application Priority Data
Nov. 8, 2019 (KR) 10-2019-0142615

(51) Int. Cl. C12N 9/50; C12N 15/70

(52) U.S. Cl. CPC C12N 9/506; C12Y 304/22044; C12N 15/70; C07K 2319/02; C07K 2319/03; C07K 2319/91

(57) ABSTRACT

The present invention relates to synthetic α-secretase (SAS) and a use thereof. According to the present invention, a synthetic α-secretase, which is a fusion protein comprising, as an active ingredient, NIa protease, a fragment thereof or a variant thereof, can inhibit the formation of amyloid β, degrade extracellularly secreted amyloid β, and degrade amyloid β internalized in cells. Therefore, the present invention allows intracellular/extracellular degradation of amyloid β, which is the cause of various diseases including Alzheimer's disease, and thus can be usable in the prevention or treatment of such diseases.

### TECHNICAL FIELD

The present invention relates to synthetic α-secretase and a use thereof.

### BACKGROUND ART

Dementia means that the gradual loss of rational thinking and the ability to engage in social activities becomes serious enough to cause an obstacle in a person's daily life. Dementia can be largely divided into dementia caused by Alzheimer's disease, Parkinson's disease, Huntington's disease, and vascular dementia caused by blockage or narrowing of blood vessels, and among these, Alzheimer's disease is the most common cause of dementia.

Drugs used to treat Alzheimer's disease can largely be classified into antioxidants, anti-inflammatory drugs, female hormones, and acetylcholinesterase inhibitors. However, most of these drugs have serious side effects, and also have limitations in that they are drugs for relieving symptoms instead of treating the underlying cause of Alzheimer's disease. Therefore, there is an urgent need to develop therapeutic agents for the fundamental treatment of Alzheimer's disease.

Alzheimer's disease is anatomically characterized by amyloid plaques generated outside of brain cells and neurofibrillary tangles (NFTs) generated within the cells. Amyloid plaques consist of a small peptide called amyloid β (Aβ), which is known to be a major cause of Alzheimer's disease.

Specifically, amyloid β, which is a major component forming amyloid plaques in the brain of Alzheimer's disease patients, is a peptide composed of 39 to 43 amino acids and is also observed in patients with Lewy body dementia, inclusion body myositis, or myopathy. Additionally, amyloid β forms aggregates coating cerebral blood vessels in patients with cerebral amyloid angiopathy. These amyloid plaques are protein folds shared by other peptides (e.g., prions) and consist of bundles of well-ordered fibrillar aggregates called amyloid fibers. The water-soluble site of amyloid β peptide, which is formed by cleavage from amyloid precursor protein (APP), acts act as a determining factor in the progression of Alzheimer's disease. The formation of this amyloid β is achieved through the degradation of amyloid precursor protein by β-secretase and γ-secretase. On the other hand, when amyloid precursor protein is degraded by α-secretase (which competes with β-secretase) instead of β-secretase, the formation of amyloid β is inhibited (LaFerla F M et al., 2007).

The α-secretase cleaves the amyloid precursor protein to produce soluble APPα (hereinafter “sAPPα”). The produced sAPPα has a neuroprotective effect and plays an important role in the survival of neurons (Christian Tackenberg et al., Molecular Brainvolume 12, 27 (2019)). In contrast, the β-secretase cleaves the amyloid precursor protein to produce soluble APPβ (hereinafter “sAPPβ”) and amyloid β. The produced sAPPβ and amyloid β are known to cause neurotoxicity. Therefore, development of β-secretase and γ-secretase inhibitors and substances capable of activating α-secretase are in progress as a method to inhibit the formation of amyloid β. Studies on amyloid β-degrading enzymes to remove already formed amyloid β in different directions are underway.

### DISCLOSURE OF INVENTION

### Technical Problem

The present invention is to provide a novel fusion protein which includes NIa protease or a fragment thereof, or a variant thereof, and a therapeutic agent for amyloid β-associated diseases prepared using the same.

### Solution to Problem

In order to achieve the object, the present invention provides a fusion protein of Formula 1 below:

(X)ₘ-(L1)ₙ-A-(L2)ₒ-(Y)ₚ-(Z)_{q} [Formula 1]

in which in Formula 1 above,

X is a signal sequence;

L1 is peptide linker 1;

A is a nuclear inclusion a (NIa) protease or a fragment thereof, or a variant thereof;

L2 is peptide linker 2;

Y is a transmembrane domain;

Z is an intracellular domain; and

m, n, o, p, and q are each 0 or 1.

Additionally, the present invention provides a polynucleotide encoding the fusion protein.

Additionally, the present invention provides an expression vector including the polynucleotide.

Additionally, the present invention provides a recombinant virus including the polynucleotide.

Additionally, the present invention provides a host cell transfected with an expression vector.

Additionally, the present invention provides a pharmaceutical composition for preventing or treating diseases caused by amyloid β, which includes the expression vector as an active ingredient.

Additionally, the present invention provides a method for preventing or treating diseases caused by amyloid β, which includes administering the composition to an individual.

### Advantageous Effects of Invention

The synthetic α-secretase (hereinafter, SAS), which is a fusion protein according to the present invention that includes NIa protease, a fragment thereof, or a variant thereof as an active ingredient, can inhibit the formation of amyloid β, can degrade amyloid β secreted extracellularly, and can also degrade amyloid β that is introduced into cells from the outside. Therefore, the present invention can be utilized for degrading amyloid β, which is the cause of various diseases including Alzheimer's disease, thereby preventing or treating these diseases.

### BRIEF DESCRIPTION OF DRAWINGS

FIG. 1 shows a drawing confirming the activity of a fragment of NIa using amyloid β as a substrate.

FIG. 2 shows a schematic drawing illustrating the structure of SAS. NIa protease is a protein present in the cytoplasm. However, SAS, which is a novel fusion protein including NIa protease, a fragment thereof, or a variant thereof, is expressed and acts in the protein secretion pathway including the endoplasmic reticulum. In particular, ss stands for a signal sequence.

FIG. 3 shows a schematic drawing illustrating the structure of a fusion protein sub(+), which includes a substrate sequence in which SAS can act, and the structure of a fusion protein sub(−), where P1 (histidine) and P2 (glutamine) are designed to prevent SAS from acting by substituting these parts with alanine.

FIG. 4 shows a drawing confirming that SAS has an α-secretase activity that specifically cleaves the amyloid β sequence using the fusion protein sub(+) and sub(−) shown in FIG. 3 as substrates through the Western blot technique.

FIG. 5 shows a schematic drawing illustrating the structure of an amyloid precursor protein from which amyloid β is derived, in which the sites cleaved by α-secretase, β-secretase, and SAS are indicated.

FIG. 6 shows a schematic drawing illustrating the structures of SAS and a few variants of SAS.

FIG. 7 shows a drawing illustrating the measurement results of activities of SAS or SAS variants through the Western blot technique using the sAPPα antibody, after transfecting HEK293T cells with a plasmid encoding an amyloid precursor protein and SAS or a variant of SAS.

FIG. 8 shows a drawing confirming the N-glycosylation of SAS within HEK293T cells using EndoH and PngF (i.e., enzymes for removing N-glycosylation) through the Western blot technique.

FIG. 9 shows a schematic drawing illustrating the positions for asparagine (N70, N115, or N253) which are the sites where the N-glycosylation occurs, and variants where N70, N115, or N253 was substituted with glutamine (Q) to prevent the occurrence of N-glycosylation.

FIG. 10 shows a drawing illustrating the measurement results of activities of SAS and various SAS variants, in which N-glycosylation was inhibited, through the Western blot technique using the sAPPα antibody.

FIG. 11 shows a drawing confirming that not only fusion proteins prepared using the NIa protease derived from TuMV, but also fusion proteins prepared using the NIa protease derived from Potato Virus Y (PVY), Sunflower Chlorotic Mottle Virus (ScMV), and Wild Potato Mosaic Virus (WpMV) can have the activity as an SAS, through the Western blot technique using the sAPPα antibody.

FIG. 12a shows a graph illustrating the open field test (OFT) results with regard to the 5XFAD mice of the NL-EGFP group, NL-SAS group, TG-EGFP group, and TG-SAS group.

FIG. 12b shows a graph illustrating the novel object recognition (NOR) test results with regard to the 5XFAD mice of the NL-EGFP group, NL-SAS group, TG-EGFP group, and TG-SAS group (* : p<0.05, ** : p<0.01).

FIG. 12c shows a graph illustrating the Morris water maze (MWM) test results with regard to the 5XFAD mice of the NL-EGFP group, NL-SAS group, TG-EGFP group, and TG-SAS group, in which the time taken to find the location of the platform for each date (* : p<0.05, ** : p<0.01, *** : p<0.005).

FIG. 12d shows a drawing illustrating the paths taken by the mice in each group in the final test on the second day of reversal of the Morris water maze (MWM) test with regard to the 5XFAD mice of the NL-EGFP group, NL-SAS group, TG-EGFP group, and TG-SAS group.

FIG. 12e shows a graph illustrating the measurement results regarding the amount of time each group of mice stayed in the original quadrant of the platform on the second day of reversal of the Morris water maze (MWM) test with regard to the 5XFAD mice of the NL-EGFP group, NL-SAS group, TG-EGFP group, and TG-SAS group (* : p<0.05, *** : p<0.005).

FIG. 13a shows a photographic image taken using a fluorescence microscope after staining the plaques in the brain tissue of a 5XFAD mouse in the TG-EGFP group using the Aβ antibody.

FIG. 13b shows a photographic image taken using a fluorescence microscope after staining the plaques in the brain tissue of a 5XFAD mouse in the TG-SAS group using the Aβ antibody.

FIG. 13c shows a graph illustrating the results of the number of plaques in the hippocampus region of the brain calculated by quantitative analysis after staining the plaques in the brain tissue of 5XFAD mice in the TG-EGFP group and the TG-SAS group using the Aβ antibody (*** : p<0.005).

FIG. 13d shows a graph illustrating the results of the proportion of area occupied by the plaques in the hippocampus region of the brain calculated by quantitative analysis after staining the plaques in the brain tissue of 5XFAD mice in the TG-EGFP group and the TG-SAS group using the A13 antibody (*** : p<0.005).

### BEST MODE FOR CARRYING OUT THE INVENTION

SAS a Fusion Protein

In one aspect, the present invention provides a fusion protein which includes NIa protease or a fragment thereof.

In a specific embodiment, the present invention provides a fusion protein which includes NIa protease or a fragment thereof; and a transmembrane domain.

In a specific embodiment, the fusion protein may be a fusion protein having the structure of Formula 1 below:

(X)ₘ-(L1)ₙ-A-(L2)ₒ-(Y)ₚ-(Z)_{q} [Formula 1]

in which in Formula 1 above,

X may be a signal sequence; L1 may be peptide linker 1; A may be a nuclear inclusion a NIa protease or a fragment thereof, or a variant thereof; L2 may be peptide linker 2; Y may be a transmembrane domain; Z may be an intracellular signaling domain; and m, n, o, p, and q may be each 0 or 1.

Specifically, the X, which is a signal sequence, may be derived from β-secretase.

The β-secretase may be a polypeptide having the amino acid sequence represented by SEQ ID NO: 1, and the gene encoding the same may be a polynucleotide having the nucleotide sequence of SEQ ID NO: 2.

Specifically, the X may be an amino acid at positions 1 to 21 in the amino acid sequence of β-secretase represented by SEQ ID NO: 1. The X may be a polypeptide having the amino acid sequence of SEQ ID NO: 3. In the fusion protein of the present invention, the X may be present or absent.

In the present invention, the L1 refers to peptide linker 1. The peptide linker 1 may be a peptide consisting of 1 to 24 amino acids. Specifically, the linker may be a peptide consisting of 1 to 24, 2 to 20, 3 to 15, or 5 to 10 amino acids, and more specifically, a peptide consisting of 3 to 24 amino acids.

In an embodiment, the linker may be a polypeptide represented by an amino acid sequence selected from the group consisting of GTDLVSIPHGPNVTVRANIAAI (SEQ ID NO: 5), DLVSIPHGPNVTVRANIAAI (SEQ ID NO: 6), DLVSIPHGPNVTVRANIA (SEQ ID NO: 7), VSIPHGPNVTVRANIA (SEQ ID NO: 8), IPHGPNVTVRANIA (SEQ ID NO: 9), IPHGPNVTVRAN (SEQ ID NO: 10), and HGPNVTVRAN (SEQ ID NO: 11), but the linker is not limited thereto. The linker merely has a role of linking these monomers, and it has a very limited effect on the structure and impact.

In a specific embodiment, the L1 may be a pro-domain of β-secretase. Specifically, the pro-domain of β-secretase may be amino acids at positions 22 to 45 (SEQ ID NO: 4) in the amino acid sequence of β-secretase represented by SEQ ID NO: 1. In the fusion protein of the present invention, the L1 may be present or absent.

The A may be NIa protease. As used herein, the term “NIa protease” has an activity of effectively degrading amyloid β. The NIa protease may be derived from the family Potyviridae. Preferably, the NIa protease derived from the family Potyviridae may be able to specifically cleave the valine-X-histidine-glutamine (VXHQ) site. In particular, the X may be any one selected from 20 amino acids. Specifically, the NIa protease may be one derived from Agropyron mosaic virus, Algerian watermelon mosaic virus, Alstroemeria mosaic virus, Alternanthera latent virus, Amaranthus leaf mottle virus, Amazon lily mosaic virus, Angelica virus Y, Apium virus Y, Arracacha mottle virus, Basella rugose mosaic virus(=Peace lily mosaic virus), Bermuda grass southern mosaic virus, Bidens mosaic virus, Bidens mottle virus, Brugmansia suaveolens mottle virus, Brugmansia mosaic virus, Canna yellow streak virus, Carrot thin leaf virus, Carrot virus Y, Catharanthus mosaic virus, Celery mosaic virus, Celery yellow mosaic virus, Chilli ringspot virus, Chilli veinal mottle virus, Chinese artichoke mosaic virus, Christmas bell potyvirus, Cocksfoot streak virus, Commelina mild mosaic virus, Cotyledon virus Y, Cypripedium virus Y, Delphinium vein clearing virus, Donkey orchid virus A, Ecuadorian rocoto virus, Endive necrotic mosaic virus, Euphorbia ringspot virus, Gloriosa stripe mosaic virus, Henbane mosaic virus, Hordeum mosaic virus, Hyacinth mosaic virus, Ipomoea vein mosaic virus, Iris mild mosaic virus, Japanese yam mosaic virus, Johnsongrass mosaic virus, Kalanchoe mosaic virus, Keunjorong mosaic virus, Konjac mosaic virus, Lettuce mosaic virus, Lupin mosaic virus, Maize dwarf mosaic virus, Malva vein clearing virus, Moroccan watermelon mosaic virus, Muscari mosaic virus, Narcissus late season yellows virus, Narcissus yellow stripe virus, Omphalodes virus Y, Ornamental onion stripe mosaic virus, Ornithogalum mosaic virus, Ornithogalum stripe mosaic virus, Ornithogalum virus 2, Panax notoginseng virus Y, Papaya leaf distortion mosaic virus, Papaya ringspot virus, Pennisetum mosaic virus, Pepper mottle virus, Pepper severe mosaic virus, Pepper veinal mottle virus, Pepper yellow mosaic virus, Peru tomato mosaic virus, Pfaffia mosaic virus, Pleione virus Y, Plum pox virus, Potato virus V, Potato virus Y, Ranunculus mild mosaic virus, Ryegrass mosaic virus, Scallion mosaic virus, Sorghum mosaic virus, Sugarcane mosaic virus, Sunflower chlorotic mottle virus, Sweet potato feathery mottle virus, Sweet potato latent virus, Sweet potato virus 2, Sweet potato virus G, Tobacco vein banding mosaic virus, Tomato necrotic stunt virus, Trillium crinkled leaf virus, Tuberose mild mosaic virus, Tuberose mild mottle virus, Turnip mosaic virus, Unnamed Verbena potyvirus, Vallota mosaic virus, Vanilla distortion mosaic virus, Verbena virus Y, Wild potato mosaic virus, Wild tomato mosaic virus, Yam mild mosaic virus, Yam mosaic virus, Zantedeschia mosaic virus, Zea mosaic virus (=Iranian Johnsongrass mosaic virus), or Zucchini yellow fleck virus, but is not limited thereto.

In a specific embodiment, the NIa protease may be from Turnip mosaic virus (TuMV). TuMV is a pathogenic plant virus in plants of the family Brassicaceae, which is infected by 40 to 50 different kinds of aphids, and the infected plants show symptoms such as chlorotic local lesions, mosaics, stains, and wrinkles. TuMV, which has no envelope, is a positive-sense single-stranded RNA virus composed of a helical capsid with an average length of 720 nm. The genome of TuMV consists of one open reading frame (ORF) of about 10 kb and is in the form of a linear monopartite.

The TuMV-derived NIa protease may be a polypeptide consisting of 243 amino acids represented by SEQ ID NO: 12. In an embodiment, in the present invention, the TuMV-derived NIa protease may be a polypeptide (SEQ ID NO: 13) consisting of 235 amino acids, in which the 1st to the 8th amino acids are excluded from the amino acid sequence represented by SEQ ID NO: 12.

In the present invention, the TuMV-derived NIa protease may have an amino acid sequence consisting of amino acids at positions 56 to 269 in the amino acid sequence of the SAS represented by SEQ ID NO: 31. Specifically, the NIa protease may be a polypeptide having an amino acid sequence represented by SEQ ID NO: 15.

Additionally, the A may be a TuMV-derived NIa protease fragment. In an embodiment, the NIa protease fragment may be in a form in which 18 to 30 amino acid residues, and specifically 20, 21, 22, 23, 24, 25, 26, 27, or 28 amino acid residues in a direction from the C-terminus to the N-terminus in the amino acid sequence of the NIa protease represented by SEQ ID NO: 13 may be consecutively deleted. In a specific embodiment of the present invention, amyloid β-degrading activity was confirmed in the NIa protease fragments (SEQ ID NOS: 14, 16, and 17, respectively), in which 20, 24, and 28 amino acid residues were deleted from the C-terminus.

In a specific embodiment of the present invention, the TuMV-derived NIa protease fragment may be a peptide consisting of 200 to 250 amino acids in the amino acid sequence of the NIa protease represented by SEQ ID NO: 12 or 13. Specifically, the NIa protease fragment may be a polypeptide having any one of the amino acid sequences represented by SEQ ID NO: 12 to SEQ ID NO: 21.

Additionally, the A may be a TuMV-derived NIa protease variant. The NIa protease variant of the present invention may be one which, while exhibiting a biological activity equivalent to that of the NIa protease of the present invention, the amino acid sequence of wild-type NIa is mutated. In the present invention, the expression “exhibiting a biological activity equivalent to NIa protease” means that even if a specific amino acid in the amino acid sequence of NIa protease is substituted, deleted, or mutated, the NIa protease still has the activity of degrading amyloid β as in wild-type NIa protease.

Such amino acid mutations are made based on the relative similarity of amino acid side chain substituents in terms of hydrophobicity, hydrophilicity, charge, size, etc. By the analysis of the size, shape, and type of the amino acid side chain substituents, it can be seen that arginine, lysine, and histidine are all positively charged residues; alanine, glycine, and serine have a similar size; and phenylalanine, tryptophan, and tyrosine have a similar shape. Therefore, based on foregoing, arginine, lysine, and histidine; alanine, glycine, and serine; and phenylalanine, tryptophan, and tyrosine may be considered as biologically functional equivalents.

Considering the mutations with the bioequivalent activity, the NIa protease variants of the present invention may be interpreted as including even the sequences showing substantial identity to those described in the sequence listing.

In a specific embodiment, the TuMV-derived NIa protease variant may be one in which one of the N-glycosylation sites of the NIa protease is substituted with another amino acid. In particular, the N may refer to asparagine (Asn), and the other amino acid may be any one amino acid selected from the group consisting of arginine (Arg), histidine (His), lysine (Lys), aspartic acid (Asp), glutamic acid (Glu), serine (Ser), threonine (Thr), glutamine (Gln), tyrosine (Tyr), alanine (Ala), isoleucine (Ile), leucine (Leu), valine (Val), phenylalanine (Phe), methionine (Met), tryptophan (Trp), glycine (Gly), proline (Pro), and cysteine (Cys).

The N-glycosylation site of the TuMV-derived NIa protease may be N15, N60, or N198 based on the amino acid sequence of SEQ ID NO: 13, but the site is not limited to these mutations as long as it has a mutation that causes the activity of NIa protease.

Additionally, the N-glycosylation site of the TuMV-derived NIa protease may be N70, N115, or N253 based on the amino acid sequence of SEQ ID NO: 31 representing SAS, but the site is not limited to these mutations as long as it has a mutation that causes the activity of NIa protease.

In the present invention, the SAS including the TuMV-derived NIa protease or a variant thereof can inhibit the formation of amyloid β, degrade extracellularly-secreted amyloid β, and also degrade amyloid β that is introduced into the cell from the outside.

In an embodiment of the present invention, in order to determine the specific effect of N-glycosylation occurring in SAS on the activity of SAS, an attempt was made to measure the effect of cleaving the amyloid β precursor protein by preparing a combination of mutations (N70Q, N115Q, or N253Q) in which asparagine (N) at the position for N-glycosylation (N70, N115, or N253) was substituted with glutamine (Q). As a result, the N70Q showed a stronger effect of cleaving the amyloid precursor protein than the conventional SAS. In particular, based on the amino acid sequence of SEQ ID NO: 31 representing SAS, the N70Q type variant was represented by SEQ ID NO: 32, the N115Q type variant by SEQ ID NO: 33, and the N253Q type variant by SEQ ID NO: 34. Additionally, based on the amino acid sequence of SEQ ID NO: 15 representing NIa protease, the N15Q type variant was represented by SEQ ID NO: 19, the N60Q type variant by SEQ ID NO: 20, and the N198Q type variant by SEQ ID NO: 21.

In the present invention, the L2 refers to peptide linker 2. The peptide linker 2 may be a peptide consisting of 1 to 50 amino acids. Preferably, the peptide linker 2 may consist of 1 to 30 amino acids or 2 to 15 amino acids. Specifically, the linker may be a peptide consisting of 1, 2, 3, 4, 5, 6, 7, 8, or 9 amino acids.

For example, the linker may be a polypeptide represented by an amino acid sequence selected from the group consisting of QTDESTLMT (SEQ ID NO: 22), TDESTLMT (SEQ ID NO: 23), QTDESTLM (SEQ ID NO: 24), DESTLMT (SEQ ID NO: 25), QTDESTL (SEQ ID NO: 26), ESTLMT (SEQ ID NO: 27), and QTDEST (SEQ ID NO: 28), but the linker is not limited thereto. The linker merely has a role of linking these monomers, and it has a very limited effect on the structure and impact.

In a specific embodiment, the L2 may be derived from β-secretase. Specifically, the peptide linker 2 may be an amino acid sequence consisting of amino acids at positions 449 to 457 in the amino acid sequence of β-secretase represented by SEQ ID NO: 1 (SEQ ID NO: 22). In the fusion protein of the present invention, the L2 may be present or absent.

The Y may be a transmembrane domain. As used herein, the term “transmembrane domain” refers to a peptide located at a position passing through the cell membrane in the structure of the proteins located on the cell membrane. The transmembrane domain can locate the NIa protease or a fragment thereof or a NIa protease variant inside the endoplasmic reticulum, Golgi apparatus, or endosome, or on the surface of a cell membrane. Additionally, the transmembrane domain has a role of linking NIa protease or a fragment thereof or a NIa protease variant to a domain that transmits an intracellular signal. The transmembrane domain may be derived from any one selected from the group consisting of β-secretase, ELAVL3, NRGN, REEP2, GAD1, PCDHA1, GFAP, S100B, FAM19A1, AQP4, and CLEC2L.

In a specific embodiment, the Y, as a transmembrane domain, may be a polypeptide represented by the amino acid sequence of SEQ ID NO: 29. In the fusion protein of the present invention, the Y may be present or absent.

In the fusion protein of the present invention, the Z may be an intracellular domain. The intracellular domain may be an intracellular signaling domain of a membrane protein.

As used herein, the term “intracellular domain” refers to a region located in the cytoplasm of a membrane protein. In a specific embodiment, the intracellular domain may be a region present in a cell as the C-terminus of the membrane protein.

As used herein, the term “intracellular signaling domain” refers to a region which transmits signals into a cell so as to induce responses (e.g., cell activation, release of cytotoxic factors, cytokine production, proliferation, etc.) when a signaling receptor on the cell surface recognizes an extracellular signaling substance.

Specifically, the intracellular domain may be derived from β-secretase. The intracellular domain may be an amino acid sequence at positions 479 to 501 in the β-secretase sequence represented by SEQ ID NO: 1, and in a specific embodiment, the intracellular domain may be a polypeptide represented by the amino acid sequence of SEQ ID NO: 30. In the fusion protein of the present invention, the Z may be present or absent.

The fusion protein of the present invention may have a structure of a fusion protein of various combinations depending on the presence or absence of X, L1, L2, Y, or Z of Formula 1 according to the present invention.

In a specific embodiment, the fusion protein of the present invention may be X-L1-A-L2-Y-Z; X-L1-A-Y-Z; X-L1-A-Y; L1-A-Y; L1-A-Y-Z; X-A -Y-Z; X-A-Y; A-Y-Z; X-A; or A-Y, but is not limited thereto.

In a specific embodiment, the fusion protein of the present invention may have a fusion protein structure of Formula 2 below, in which a TuMV-derived β-secretase signal sequence, a TuMV-derived NIa protease, a transmembrane domain, and an intracellular domain are fused.

X-A-L2-Y-Z [Formula 2]

In the present invention, the fusion protein was expressed as SAS2. SAS2 having the structure of Formula 2 may be a polypeptide represented by the amino acid sequence of SEQ ID NO: 39.

Additionally, the fusion protein of the present invention may have a structure of Formula 3 or Formula 4 below, in which the signal sequence of β-secretase and the pro domain (pro) represented by peptide linker 1 are fused with the NIa variant (D128N) or the NIa variant (N70Q).

X-L1-A(D128N)-L2-Y-Z [Formula 3]

X-L1-A(N70Q)-L2-Y-Z [Formula 4]

In particular, the NIa variant (D128N) is a negative variant in which the activity is removed by substituting the aspartic acid (D) at position 128 in the catalytic triad with asparagine (N). This variant, based on the amino acid sequence of SEQ ID NO: 15 representing the NIa protease, corresponds to D73N (SEQ ID NO: 18). SAS^{m} having the structure of Formula 3 may be a polypeptide represented by the amino acid sequence of SEQ ID NO: 35. Additionally, the NIa variant (N70Q) was prepared to measure its effect on SAS performance by inactivating one of the N-glycosylation sites of SAS to prevent glycosylation. The SAS^{N70Q} having the structure of Formula 4 above may be C represented by the amino acid sequence of SEQ ID NO: 32.

Additionally, the fusion protein of the present invention may have a structure of Formula 5 below, in which the intracellular signaling domain is removed.

X-L1-A-L2-Y [Formula 5]

Additionally, the fusion protein of the present invention may have a structure of Formula 6 below, in which the transmembrane domain Y and the intracellular domain Z are absent.

X-L1-A [Formula 6]

In particular, the fusion protein (SEC) of Formula 6 above was prepared to be secreted extracellularly by removing the transmembrane domain therein. The SAS' having the structure of the Formula 6 may be a polypeptide represented by the amino acid sequence of SEQ ID NO: 37.

Additionally, the fusion protein of the present invention may have a structure of Formula 7 below including a GPI targeting signal.

X-L1-A-L2-Y(GPI targeting signal) [Formula 7]

Formula 7 above is in a form in which a signal sequence of β-secretase, a prodomain represented by peptide linker 1, NIa protease, and peptide linker 2 are fused with a transmembrane domain Y. In particular, the transmembrane domain may be a GPI targeting signal that is not derived from β-secretase. The SAS^{GPI} having the structure of the Formula 7 may be a polypeptide represented by the amino acid sequence of SEQ ID NO: 38.

In another specific embodiment, the present invention, among the various structures of SAS prepared for the purpose of confirming SAS activity, provides SAS3 which was prepared in such a manner that a murine immunoglobulin Kappa chain V-J2-C signal sequence unrelated to β-secretase was fused to the N-terminus of NIa and a PDGFR transmembrane domain was fused to the C-terminus of NIa. The may be a polypeptide represented by the amino acid sequence of SEQ ID NO: 40.

Additionally, the present invention provides SAS4 prepared by fusing the N-terminus of US9 to the C-terminus of NIa using a type 2 transmembrane domain. The SAS4 may be a polypeptide represented by the amino acid sequence of SEQ ID NO: 41.

Polynucleotide Encoding Fusion Protein

In another aspect, the present invention provides a polynucleotide encoding a fusion protein according to the present invention.

As used herein, the term “polynucleotide” refers to deoxyribonucleic acid (DNA), ribonucleic acid (RNA), or a DNA/RNA hybrid. The polynucleotide may be single-stranded or double-stranded and may be recombinant, synthetic, or isolated. The polynucleotide includes pre-messenger RNA (pre-mRNA), messenger RNA (mRNA), RNA, genomic RNA (gRNA), plus-stranded RNA (RNA (+)), minus-stranded RNA (RNA (−)), synthetic RNA, synthetic mRNA, genomic DNA (gDNA), PCR amplified DNA, complementary DNA (cDNA), synthetic DNA, or recombinant DNA, but the polynucleotide is not limited thereto.

The polynucleotide may consist of a nucleotide sequence represented by SEQ ID NO: 42. Additionally, the polynucleotide may have about 80%, 90%, 95%, or 99% or more homology to the nucleotide sequence of SEQ ID NO: 42 as long as it can encode the protein of SEQ ID NO: 31. Additionally, the polynucleotide can be codon-optimized.

Vectors Expressing SAS

Additionally, the present invention provides an expression vector which includes the polynucleotide according to the present invention. The polynucleotide is the same as described above.

The polynucleotide can be prepared, engineered, expressed, and delivered using any of a variety of established techniques known and available in the art. For the expression of a target a fusion protein, a polynucleotide encoding the fusion protein may be inserted into an appropriate vector.

As the vector, a variety of vectors known in the art may be used, and depending on the type of the host cell to produce the antigen receptor, expression control sequences (e.g., promoters, terminators, enhancers, etc.), sequences for membrane targeting or secretion, etc. can be appropriately selected and variously combined according to the purpose. The vector of the present invention includes a plasmid vector, a cosmid vector, a bacteriophage vector, a viral vector, etc., but is not limited to. Suitable vectors include a signal sequence for membrane targeting or secretion or a leader sequence, in addition to expression control elements (e.g., a promoter, an operator, an initiation codon, a stop codon, a polyadenylation signal, an enhancer, etc.), and may be prepared in various ways depending on the purpose.

Preferably, the vector may be a viral vector, and the viral vector may be derived from retroviruses, lentiviruses, adenoviruses, adeno-associated viruses (AAV), herpesviruses, poxviruses, baculoviruses, papillomaviruses, vaccinia viruses, and parvoviruses. In an embodiment of the present invention, a lentiviral vector was used.

Viruses Containing Nucleic Acid Encoding SAS

The present invention provides a recombinant virus comprising the polynucleotide according to the present invention. The polynucleotide is the same as described above, and the recombinant virus may be prepared using a plasmid loaded with the polynucleotide according to the present invention.

The recombinant virus may be any one derived from retroviruses, lentiviruses, adenoviruses, adeno-associated viruses (AAV), herpesviruses, poxviruses, baculoviruses, papillomaviruses, vaccinia viruses, and parvoviruses, but is not limited thereto.

In the present invention, the fusion protein that can be expressed by the virus may be a fusion protein in which a signal sequence is removed. In a specific embodiment, the fusion protein may be L1-A-L2-Y-Z; L1-A-Y-Z; L1-A-Y; L1-A-Y; A-Y-Z; A-Y; or A, but is not limited thereto.

Cells Expressing SAS

The present invention provides a host cell transfected with an expression vector according to the present invention. In particular, the expression vector is the same as described above. The host cell may be any one selected from the group consisting of E. coli, CHO cells, and HEK293 cells, but is not limited thereto. The method of introducing the expression vector of the present invention into a cell can be performed through various methods known in the art.

The methods for introducing the expression vector into cells may be performed using the methods known in the art, for example, transient transfection, microinjection, transduction, cell fusion, calcium phosphate precipitation, liposome-mediated transfection, DEAE Dextran-mediated transfection, polybrene-mediated transfection, electroporation, gene gun, or other known methods for introduction of a nucleic acid into cells (Wu et al., J. Bio. Chem., 267:963-967, 1992; Wu and Wu, J. Bio. Chem., 263: 14621-14624, 1988). However, the methods are not limited thereto.

Transfected host cells may be proliferated ex vivo after the introduction of an expression vector. In a specific embodiment, the transfected host cells may be cultured at least for about 1, 2, 3, 4, 5, 6, 7, 8, 9, 10, 11, 12, 13, or 14 days to proliferate, and preferably, may be cultured for 12 to 14 days.

The methods for confirming whether an expression vector has been well introduced into the host cell include, for example, molecular biological assays well known to those skilled in the art, for example, Southern and Northern blotting, RT-PCR and PCR; biochemical assays, for example, detection of the presence or absence of a particular peptide by immunological methods (e.g., ELISAs and Western blotting).

In the present invention, the fusion protein that can be expressed by the host cell may be a fusion protein in which a signal sequence is removed. In a specific embodiment, the fusion protein may be L1-A-L2-Y-Z; L1-A-Y-Z; L1-A-Y; A-Y-Z; A-Y; or A, but is not limited thereto.

Pharmaceutical Composition Containing a Vector Expressing SAS

In another aspect, the present invention provides a pharmaceutical composition for preventing or treating diseases caused by amyloid β containing the expression vector as an active ingredient.

The pharmaceutically acceptable carriers to be contained in a pharmaceutical composition of the present invention are those commonly used in formulation, which include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, etc., but the pharmaceutically acceptable carriers are not limited thereto. The pharmaceutical composition of the present invention may further contain a lubricant, a wetting agent, a sweetening agent, a flavoring agent, an emulsifying agent, a suspending agent, a preservative, etc. in addition to the components described above.

The pharmaceutical composition of the present invention is preferably administered parenterally, for example, may be administered through intravenous administration, subcutaneous administration, or local administration. A suitable dose of the pharmaceutical composition of the present invention varies depending on factors (e.g., a formulation method, an administration method, age, weight, and sex of the patient, degree of disease symptoms, food, administration time, administration route, excretion rate, and response sensitivity) and an ordinarily skilled physician can easily determine and prescribe a dose effective for the intended treatment. In general, the daily dose of the pharmaceutical composition of the present invention may be 0.0001 mg/kg to 100 mg/kg.

The pharmaceutical composition of the present invention may be formulated using a pharmaceutically acceptable carrier and/or excipient according to a method that can easily be performed by those skilled in the art to which the present invention pertains, to be prepared in a unit dose form or prepared by incorporation into a multi-dose container. In particular, the formulation may be in the form of a solution, suspension, or emulsion in oil or aqueous medium, or may be in the form of an extract, powder, granule, tablet, or capsule, and may additionally include a dispersant or stabilizer.

The disease caused by the amyloid β may be a neurodegenerative disease, and specifically, may be any one selected from the group consisting of Alzheimer's disease, mild cognitive impairment (MCI), mild-to-moderate cognitive impairment, vascular dementia, cerebral amyloid angiopathy, hereditary cerebral hemorrhage, senile dementia, Down syndrome, inclusion body myositis, age-related macular degeneration, and conditions associated with Alzheimer's disease

In the present invention, Alzheimer's disease may include acquired or congenital Alzheimer's disease. Additionally, conditions associated with Alzheimer's disease may include hypothyroidism, cerebrovascular disease, cardiovascular disease, memory loss, anxiety, behavioral dysfunction, neurological or psychological conditions.

The behavioral dysfunction includes apathy, agitation/aggression, depression, irritability/instability, anxiety, or psychosis, but is not limited to. The neurological conditions include Huntington's disease, amyotrophic lateral sclerosis, acquired immunodeficiency syndrome, Parkinson's disease, aphasia, apraxia, agnosia, Pick disease, Lewy body dementia (DLB), altered muscle tone, seizures, sensory loss, visual field deficits, incoordination, gait disturbance, transient ischemic attack or stroke, transient alertness, attention deficit, frequent falls, syncope, susceptibility to tranquilizers, normal pressure hydrocephalus (NPH), subdura hematoma, brain tumor, post-traumatic brain injury, and posthypoxic damage, but the neurological conditions are not limited thereto. The psychological conditions include depression, delusion, illusion, hallucination, sexual dysfunction, weight loss, psychosis, sleep disturbance, insomnia, behavioral disinhibition, degeneration of vision, suicidal ideation, depressive disorder, irritability, anhedonia, seclusion loneliness, and excessive guilt, but the psychological conditions are not limited thereto.

Pharmaceutical Composition Containing Virus Including Nucleic Acid Encoding SAS

Additionally, the present invention provides a pharmaceutical composition for preventing or treating diseases caused by amyloid β containing the recombinant virus according to the present invention as an active ingredient. In particular, the polynucleotide, recombinant virus, diseases caused by amyloid β, ingredients of the pharmaceutical composition, and administration route are the same as described above.

A preferred dose of the pharmaceutical composition containing the recombinant virus of the present invention varies depending on the condition and weight of the individual, the degree of disease, the drug form, and the route and duration of administration, and may be appropriately selected by those skilled in the art. Specifically, the preferred dose may be to administer 1×10⁵ to 1×10⁸ virus particles, virus units having infectivity (tissue culture infectious dose: TCID50), or plaque forming units (pfu) to a patient, and preferably, to administer 1×10⁵, 2×10⁵, 5×10⁵, 1×10⁶, 2×10⁶, 5×10⁶, 1×10⁷, 2×10⁷, 5×10⁷, 1×10⁸, 2×10⁸, 5×10⁸, 1×10⁹, 2×10⁹, 5×10⁹, 1×10¹⁰, 5×10¹⁰, 1×10¹¹, 5×10¹¹, 1×10¹², 1×10¹³, 1×10¹⁴, 1×10¹⁵, 1×10¹⁶, 1×10¹⁷ or higher virus particles, viral units having infectivity, or plaque forming units, and may include various numerical values and ranges therebetween. Additionally, the viral dose is 0.1 mL, 1 mL, 2 mL, 3 mL, 4 mL, 5 mL, 6 mL, 7 mL, 8 mL, 9 mL, 10 mL or greater, including all of the values and ranges therebetween.

Unlike the NIa protease (i.e., a cytoplasmic protein), the fusion protein according to the present invention is a membrane protein, which can exist in a protein secretion pathway including the endoplasmic reticulum and the Golgi apparatus, cell membranes, extracellular-, and protein endocytosis pathway including endosomes and lysosomes. In the present invention, since the fusion protein can cleave an amyloid precursor protein at a site similar to α-secretase, it was named synthetic α-secretase (SAS). SAS can inhibit the formation of amyloid β, degrade extracellularly-secreted amyloid β, and degrade amyloid β that enters cells from the outside. Therefore, the SAS of the present invention can be utilized as a therapeutic agent for various diseases induced by amyloid β.

### MODE FOR THE INVENTION

Hereinafter, the present invention will be described in detail by the following Examples. However, the following Examples are merely illustrative of the present invention, and the present invention is not limited thereto.

### I. Experimental Preparation and Experimental Methods

### Experimental Method 1. Purification of NIa Protease Purification and Activity Measurement Using Aβ

For in vitro cleavage experiments, 0.5 μM purified NIa was mixed with 2.5 μM monomeric or oligomeric Aβ and buffer (20 mM HEPES (pH 7.4), 10 mM KCl, and 10 mM MgCl₂) and reacted at 25° C. for 1 hour or 2 hours. Thereafter, Western blot was performed using an anti-A13 antibody (4G8) in 15% polyacrylamide gel electrophoresis (PAGE).

### Experimental Method 2. Aβ Oligomerization

The synthesized A1342 peptide powder was dissolved in 1,1,1,3,3,3-hexafluoroisopropanol (Fluka) to a concentration of 1 mM, evaporated for one day, and stored. Thereafter, the Aβ peptide was dissolved in DMSO to a concentration of 2.5 mM and subjected to sonication for 10 minutes. After dissolving the Aβ in DMEM to a concentration of 100 μM, the oligomers were synthesized by maintaining the resultant at 4° C. for 24 hours.

### Experimental Method 3. Preparation of HEK293T Cells

Human Embryonic Kidney (HEK) 293T cells were grown in DMEM (Hyclone) medium containing 10% FBS, 100 μg/mL penicillin, and 100 μg/mL streptomycin, and used in the experiment. In most cases, a plasmid vector expressing the APP751 isoform was used as a substrate, and a plasmid expressing a protease was co-transfected using Lipofectamin 2000 (Invitrogen).

### Experimental Method 4. Western Blot

When extracting proteins from the cell culture medium, 1 mL of the culture medium was pre-extracted before separation of cell proteins, and then dissolved in the sample buffer by adjusting the concentration. HEK293T cells were washed twice with PBS, dissolved in RIPA buffer containing a protease inhibitor, and proteins were extracted through sonication. The extracted proteins were quantified by the BCA method, and the proteins in the same amount (20 μg) were electrophoresed on 10% or 12% SDS-PAGE for 2 hours. Then, the resultant was transferred to a PVDF membrane. After blocking the PVDF membrane using 5% non-fat milk powder, Western blot was performed by reacting with an appropriate antibody (anti-myc (9B11), anti-A13 (4G8), anti-sAPPα (2D3), and anti-V5 antibodies were diluted 1:1,000 and used). The primary antibody reaction was performed overnight at 4° C. For the secondary antibody reaction, HRP-conjugated anti-mouse or anti-rabbit antibody was reacted at 1:10,000, and then washed with TBST three times. Thereafter, the presence or absence of the protein was determined by fluorescence through the ECL reaction.

### Experimental Method 5. Treatment of Enzyme for Removing N-Glycosylation

After transfecting HEK293T cells with SAS, whole proteins were extracted. Thereafter, the extracted proteins were denatured by boiling in 2 μL of denaturation buffer (10×) and 20 μL of water at 100° C. for 10 minutes. 4 μL of buffer (10×), 4 μL of 10% NP-40, and 2 μL of an enzyme for removing N-glycosylation (PNGaseF or EndoH) were added and reacted in a total volume of 40 μL at 37° C. for 1 hour. To confirm the size of SAS, Western blot was performed using an anti-myc antibody (Cell signaling, #2272).

### Experimental Method 6. Amyloid Precursor Protein (APP)

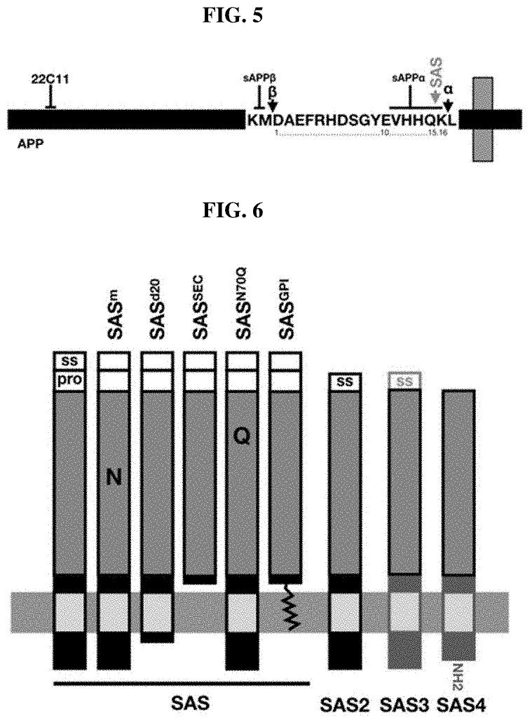

FIG. 5 briefly illustrates APP that generates A13, which is one of the causative agents of Alzheimer's disease in humans. Unlike β-secretase that produces amyloid β, α-secretase, which is known to have a neuroprotective action, is located in the middle of Aβ and acts predominantly to form sAPPα, and as a result, the formation of Aβ is reduced. Although SAS is not the exact position of α-secretase, it can produce an effect similar to that of α-secretase by cleaving between glutamine-lysine located one amino acid upstream. Additionally, since SAS does not share substrate specificity with α-secretase, problems caused by overexpression of α-secretase can be prevented. In the present invention, the activity of SAS was measured using the sAPPα antibody, and the antibody, as the antibody acting only on the C-terminus newly created by cleavage, effectively showed α-secretase activity.

### Experimental Method 7. Preparation of 5XFAD Mice and Gene Transfer Using Adeno-Associated Virus 9 (AAV9)

5XFAD mice for Alzheimer's model were purchased from Jackson Lab and then used by crossbreeding. Both negative littermate (NL) and transgenic (TG) mice obtained through crossbreeding were used in the experiment. A virus was injected into the mice using stereotaxic injection at the age of 4 months, their behavioral experiments were performed at the age of 6 months, and their brain tissue was extracted by euthanasia at the age of 9 months. As for the virus, AAV9-EGFP expressing green fluorescent protein and AAV9-SAS expressing SAS were used in the experiment. A total of 4 experimental groups were prepared by injecting EGFP and SAS viruses into NL and TG mice. For each experimental group, 7 mice in the NL-EGFP group, 6 mice in the NL-SAS group, 6 mice in the TG-EGFP group, and 8 mice in the TG-SAS group were used. For virus injection, after anesthesia with ketamine and xylazine, the mice were fixed onto a stereotaxic frame, their scalp was incised, and a hole was drilled into the skull at the location where the virus was to be injected. 10 μL of each virus was injected into both lateral ventricles of cerebrum. The drug infusion rate was set to 2 μL/min, and after waiting for 15 minutes upon completion of the injection, the needle was slowly withdrawn.

### Experimental Method 8. Open Field Test

Two days before this experiment, the mice were moved to the room where the experiment was conducted and stabilized. The day before the experiment, the mice were acclimatized by placing them in the center of an opaque test box of 36 cm*36 cm*40 cm (width*length*height) and allowing them to move freely for 5 minutes. On the day of the experiment, the mice were placed in the center of the test box and the total movement distance was measured by recording their movement for 20 minutes.

### Experimental Method 9. Novel Object Recognition Test

On the first day, two identical objects were placed at regular intervals in the center of an opaque test box of 36 cm*36 cm*60 cm (width*length*height), and a mouse was placed in the center of the objects and allowed to move freely for 8 minutes. The next day, one of the two objects was changed to another object, and the mouse was placed in the middle of the objects, and the time taken for the mouse to search for each object was measured for 8 minutes. Preference index (%) was measured through the equation (time taken to search for a new object)/(time taken to search for all objects)*100.

### Experimental Method 10. Morris Water Maze Test

Two markers were attached to the wall of a circular pool of water with a diameter of 120 cm at regular intervals, and a platform was placed 1.5 cm below the water surface near one of them so that mice could climb. The time taken for the mouse to climb the platform within 60 seconds was analyzed by placing the mouse in a different quadrant except for the place where the platform was located 4 times a day for 4 days. When the mouse arrived at the platform or the mouse was transferred to the platform, if it did not climb onto the platform within the time limit, the mouse was left on the platform for 10 seconds to memorize the location. On the fifth day, the platform was removed and the mouse was allowed to swim freely for 120 seconds, and the time and number of times it reached each quadrant and the platform were measured. On the 7th and 8th days, after changing the position of the platform in the opposite direction, the mouse was released from the position where the platform was located at the beginning, and the time for the mouse to find the position of the changed platform within 60 seconds 4 times a day was measured.

### Experimental Method 11. Immunohistochemical Analysis for Observation of Amyloid Plaques

After euthanizing the mouse, it was subjected to perfusion using PBS, and the brain was extracted. Thereafter, the tissue was fixed with 4% PFA for one day, washed with PBS for one day, and stored in 30% sucrose for 3 to 5 days. The entire process was performed at 4° C. Then, the tissue was embedded with an OCT compound, and frozen sections were prepared using a cryotome. Thereafter, the tissue was washed with PBS, fixed in 4% PFA for 20 minutes, subjected to permeabilization using 0.1% Triton X-100 solution, and blocked using 5% goat serum and a 5% BSA solution. Then, antigen retrieval was performed using a 0.1 M citrate [pH 6.0] solution, and the Aβ antibody (MOAB-2) was diluted 1:1,000 and reacted overnight. After washing with PBS, the Alexa 594-conjugated anti-mouse antibody was diluted 1:1,000 and Hoechst 33342 dye was diluted 1:2,000, and they were reacted for 4 hours. The resultant was washed with PBS and mounted. Then, the images of the hippocampus of the brain were obtained using a fluorescence microscope, and the hippocampus was subjected to quantitative analysis was performed sing ImageJ software (https://imagej.nih.gov/ij/).

### Example 1. Preparation of SAS

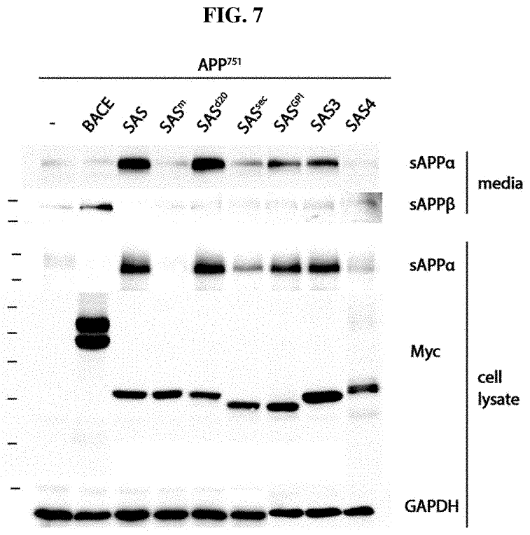

SAS was prepared by fusing the N-terminus and C-terminus of NIa with the pro domain and the transmembrane domain of β-secretase. In order to find effective SAS activity, various versions of SAS were prepared through gene synthesis (https://www.idtdna.com), and the experiment was performed by inserting the prepared SAS into the pcDNA4 vector suitable for intracellular expression using EcoRI and XhoI. SAS (SAS^{d20}, SAS^{SEC}, SAS^{GPI}, etc.) was prepared by fusion of signal sequences and prodomains with NIa, and SAS2 was prepared using only a β-secretase signal sequence (signal sequence, ss). SAS3 was prepared together with NIa by linking the V-J2-C signal sequence of the murine immunoglobulin Kappa chain, which is independent of β-secretase, to the N-terminus while linking the PDGFR transmembrane domain to the C-terminus. SAS4, which is a type2 transmembrane domain, was prepared by fusing the N-terminus of US9 with NIa. The SAS structures are shown in FIG. 6, and their activity is shown in FIG. 7. As shown in FIG. 7, among the various versions of SAS, the SAS^{d20} activity was the most outstanding.

In order to study the properties of SAS, various variant forms were prepared. SAS^{m} is a negative variant (negative mutant) of the catalytic triad of NIa in which aspartic acid (D) is replaced with asparagine (N) to abolish activity. SAS^{d20}, which is also a SAS fragment, was prepared to confirm the effect of the localization signal of β-secretase on SAS activity by removing 20 amino acids corresponding to the C-terminus of β-secretase. In particular, the SAS' may be a polypeptide represented by the amino acid sequence of SEQ ID NO: 36. Meanwhile, in the intracellular domain corresponding to Z of the fusion protein according to the present invention, 1, 2, 3, 4, 5, 6, 7, 8, 9, 10, 11, 12, 13, 14, 15, 16, 17, 18, 19, or 20 amino acid residues may be deleted consecutively. In the case of SAS^{SEC}, it was prepared to be secreted extracellularly by removing the transmembrane domain, and through SAS^{GPI}, the activity of SAS was tested after binding to the cell membrane by attaching a GPI targeting signal thereto although the transmembrane domain was removed. Finally, by way of using SAS^{N70Q}, one of the N-glycosylation sites of SAS was inactivated to prevent glycosylation, and the effect on SAS performance was measured.

### II. Confirmation of SAS Activity

### Experimental Example 1. Confirmation of Amyloid β-Degrading Activity of NIa and Fragments Thereof

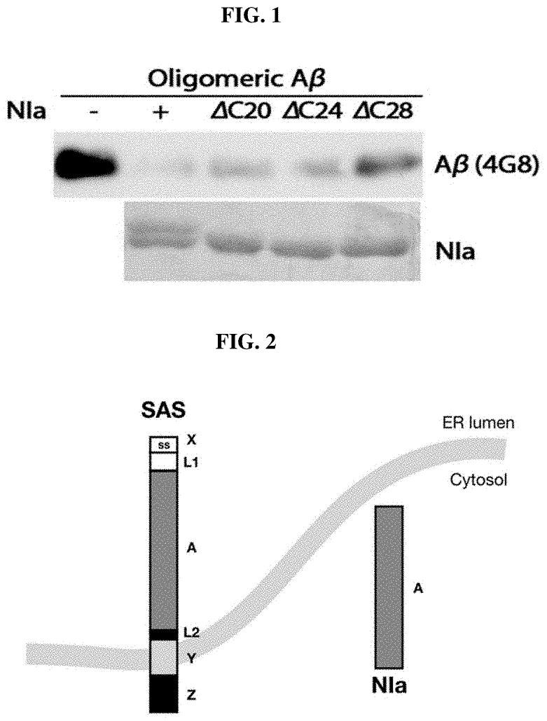

After expressing and purifying TuMV-derived NIa protease and various fragments in E. coli, their protease activity was confirmed using amyloid β as a substrate. The results of examining the amount of amyloid β under each condition are shown in FIG. 1. As shown in FIG. 1, it was confirmed that amyloid β disappeared after treatment with NIa and its fragment for 1 hour. Additionally, as self-cleavage at the C-terminus was reported in several studies on TuMV NIa, self-cleavage activity was confirmed by the appearance of two bands after protein purification in this experiment.

Experiments were performed to determine whether the protease in which the self-cleavage occurred also had its activity and whether the activity to degrade amyloid β was maintained even when more amino acids were removed. As a result, it was confirmed that NIaΔ20 (in which 20 amino acids were removed from the C-terminus) and NIaΔ24 (in which 24 amino acids were removed) showed activity similar to normal NIa, and NIaΔ28 (in which 28 amino acids were removed) showed significantly reduced activity (FIG. 1).

### Experimental Example 2. Measurements of SAS Activity

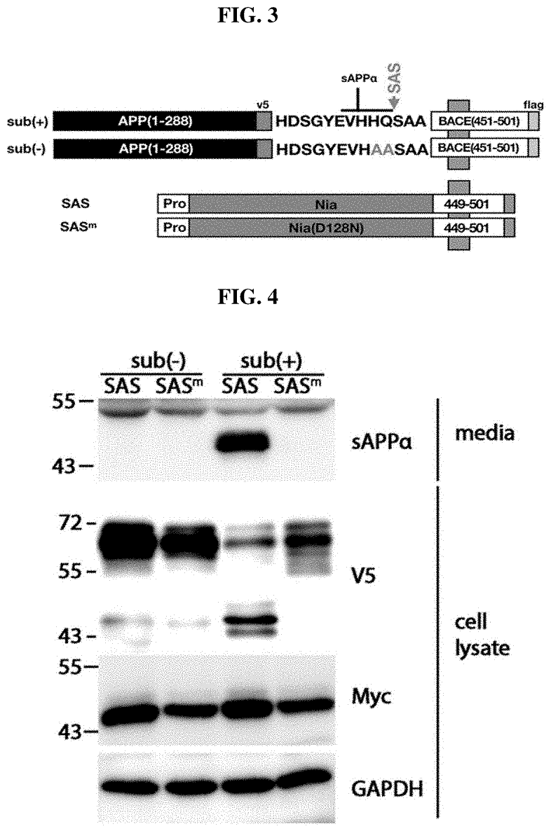

To measure the SAS activity in cells, substrate sub (+) and substrate sub (−) designed to prevent SAS from acting on the substrate were prepared. In order to block the results from being disturbed by α-secretase and γ-secretase that may exist in the cell, the substrate sequence that can act on α-secretase and γ-secretase was removed from sub (+), and the sequence optimized for SAS VXHQ/S was used. In sub (−), both the P1 (histidine) and P2 (glutamine) moieties were substituted with alanine so that SAS cannot function. For the N-terminal part, an APP N-terminus with an appropriate size was used so that it could be secreted out of the cell. Additionally, for easy detection of SAS activity, the V5 epitope was placed at the N-terminus and the Flag epitope was placed at the C-terminus. As the C-terminal portion, the C-terminus of β-secretase was used as in SAS so that the protein containing the substrate could be located at the same location in the cell as the SAS (FIG. 3). The activity of SAS confirmed by the substrate is shown in FIG. 4.

### Experimental Example 3. Confirmation of SAS Activity in HEK293 Cells

Human kidney-derived cells (HEK293T cells) were co-transfected with APP (a substrate) and vectors expressing respective proteases, and then SAS activity was measured by Western blot using the sAPPα antibody. The results are shown in FIG. 7.

As shown in FIG. 7, when only APP was transfected, the sAPPα band did not appear in the media or in the cell lysate, thus confirming that there was no or weak intrinsic α-secretase activity. Additionally, when β-secretase alone was acted on APP, it did not show an effect of increasing sAPPα, but the SAS prepared by fusion showed a distinct sAPPα band in the media and in the cell lysate. In particular, the activity of β-secretase was confirmed by the sAPPβ band.

However, in the case of the negative variant, SAS^{m}, the sAPPα band did not appear, thus confirming that the sAPPα band appeared due to the catalytic activity of SAS. When the 20 amino acids at the C-terminus, which are thought to be important for the intracellular localization of SAS, were removed (SAS^{d20}), the activity of SAS was not significantly affected. From this result, it was found that SAS activity is occurring in the process of being secreted into the cell membrane through the endoplasmic reticulum and Golgi apparatus. Considering that when SAS was prepared to be secreted immediately (SAS^{sec}) by removing the transmembrane domain, its activity was significantly reduced, whereas when it was placed again on the cell membrane through GPI (SAS^{GPI}), its activity was restored, it was found that SAS activity mainly occurs near the cell membrane.

Although the activity was the best when the protein sequence of β-secretase was used, significant activity was also shown when a sequence derived from another protein was used (SAS3), thus confirming that the N-terminus and C-terminus of β-secretase do not significantly affect the activity of SAS. In the case of SAS4, which was designed to have the N-terminus positioned inside the cell, did not increase sAPPα. Therefore, it was found that the topology of SAS is important (FIG. 7).

### Experimental Example 4. Confirmation of N-Glycosylation of SAS and Effect of Glycosylation on Activity

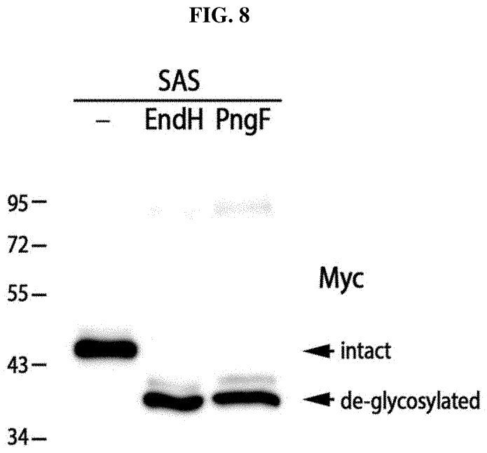

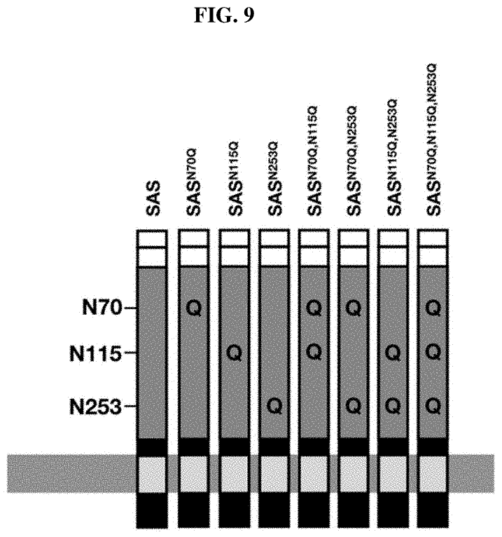

EndoH and PNGaseF (PngF), which are enzymes capable of specifically removing N-glycosylation, were reacted with SAS expressed in HEK293T cells, followed by Western blot. The results are shown in FIG. 8. As shown in FIG. 8, both EndoH and PNGaseF acted on SAS to significantly reduce their size, and considering that the reduced size was almost identical to 37 kD estimated by the amino acid sequence, it was confirmed that SAS was being N-glycosylated in the cell.

As a result of confirming the protein sequence of SAS, N70, N115, and N253 were identified as positions capable of N-glycosylation. It has been reported that when TEV NIa, which is an intracellular protease similar to TuMV, was secreted out of the cell, glycosylation, which did not occur in the original cell, proceeded, and as a result, it affected the activity of TEV Nia (Cesaratto et al., 2015). Therefore, in order to examine how glycosylation in SAS affects the activity of SAS, a combination of mutants in which asparagine at the N-glycosylation position was substituted with glutamine were prepared, allowed to express together with APP, and their APP-cleaving activity was measured.

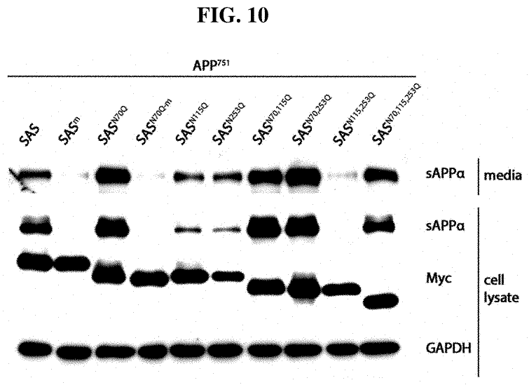

Surprisingly, the first glycosylation site mutation, SAS^{N70Q}, showed stronger activity than the original SAS. Such an effect was also seen in SAS^{N70Q,N115Q}, SAS^{N70Q,N253Q}, etc. which include N70Q, but the activity was halved or disappeared in the SAS^{N70Q,N115Q,N253Q} mutants in which glycosylation was completely eliminated. From these results, it could be speculated that the first glycosylation (N70) had an effect to interfere with the activity of SAS, and the other two glycosylation (N115, N253) were neutral or helpful for the activity (FIG. 10).

### Experimental Example 5. SAS Preparation and Activity Measurement Using NIa Protease Derived from Potyvirus Other than TuMV

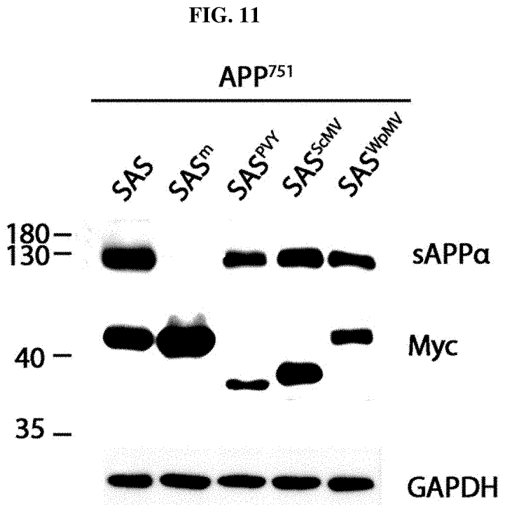

It was expected that the NIa protease of other viruses belonging to the genus potyvirus could be used for SAS production, in addition to the TuMV-derived NIa protease. In particular, it was expected that SAS could be prepared using other potyvirus-derived Nia proteases capable of specifically cleaving the valine-X-histidine-glutamine (V-X-H-Q) region, in a similar manner to TuMV-derived NIa protease. As a result of analyzing the potyvirus genome, about one hundred potyvirus NIa proteases were included in this category. In order to test this possibility, part of the N-terminus and the C-terminus were removed from each NIa protease, which was derived from Potato Virus Y (PVY, SEQ ID NO: 43), Sunflower cholorotic Mottle virus (ScMV, SEQ ID NO: 45), and Wild Potato mosaic virus (WpMV, SEQ ID NO: 47) (i.e., PVY NIa NΔ8CΔ26, SEQ ID NO: 44; ScMV NIa NΔ8CΔ26, SEQ ID NO: 46; and WpMV NIa NΔ8CΔ28, SEQ ID NO: 48) to suit SAS, and SAS was prepared as shown in FIG. 2 using the same. These new SASs were named SAS^{PVY} (SEQ ID NO: 49), SAS^{ScMV} (SEQ ID NO: 50), and SAS^{WpMV} (SEQ ID NO: 51), respectively. After co-transfection of these SASs and APP into HEK293T cells, the activity of these SASs was measured by Western blot using the sAPPα antibody. As shown in FIG. 11, these SASs showed APP cleavage activity similar to that of SAS which was prepared using TuMV-derived NIa protease.

### Experimental Example 6. Confirmation of Memory Recovery Effect of SAS in 5XFAD Mice

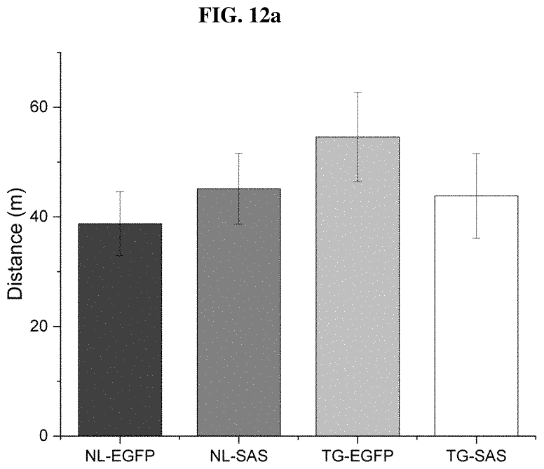

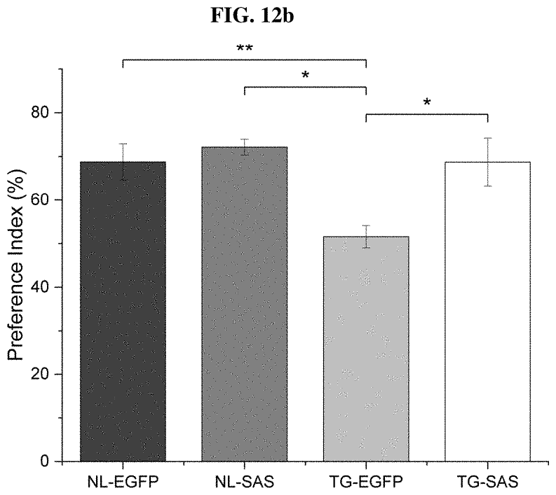

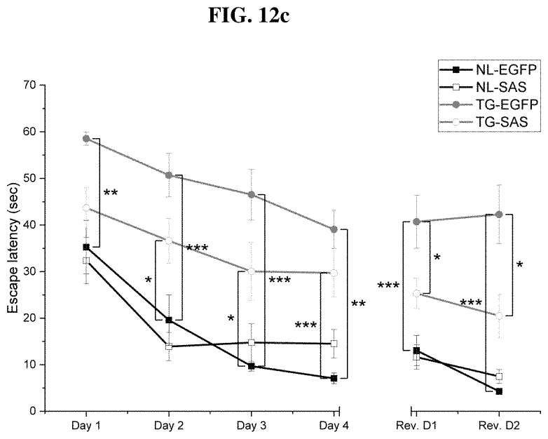

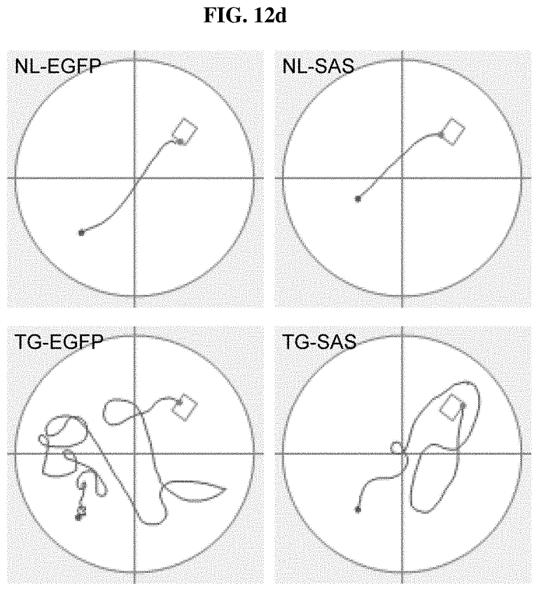

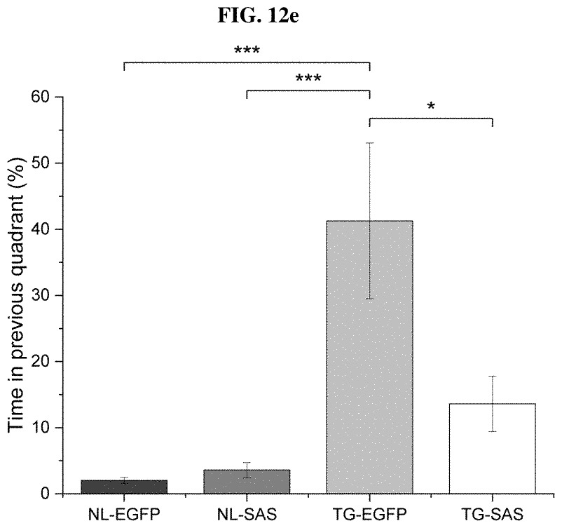

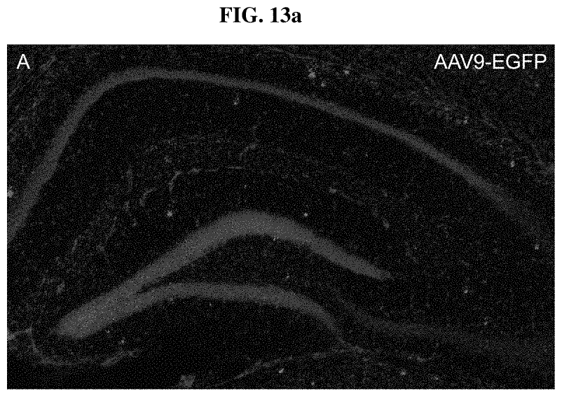

After expressing EGFP and SAS in NL and TG mice using AAV9 virus, respectively, novel object recognition (NOR) and Morris water maze (MWM) behavioral experiments were performed to observe memory ability. The results are shown in FIGS. 12a to 12e.

Referring to FIG. 12a, as a result of confirming that basic motility was normal by measuring the distance the mouse freely moved for a certain period of time with the open field test (OFT), SAS showed no effect on the basal exercise ability of the mice. Additionally, through FIG. 12b, the memory ability for a new object was observed by measuring the ratio of the search time for the existing object and the new object, and thereby confirmed that the memory ability for a new object, which had been decreased in the TG-EGFP group, was restored by expressing SAS. Additionally, reviewing FIG. 12c, it was confirmed that although the TG-EGFP group showed a difficulty in learning the platform position, this learning ability was restored through SAS expression. Further, through the reversal test of FIG. 12c (a test with the platform position reversed), FIGS. 12d, and 12e, it was confirmed that cognitive flexibility was significantly restored in the TG-SAS group when the platform position was changed compared to the TG-EGFP group. That is, through this experiment, it was confirmed that the expression of SAS using AAV9 had a therapeutic effect to restore the memory loss of 5XFAD mice.

### Experimental Example 7. Confirmation of Plaque Removal Activity of SAS in 5XFAD Mice

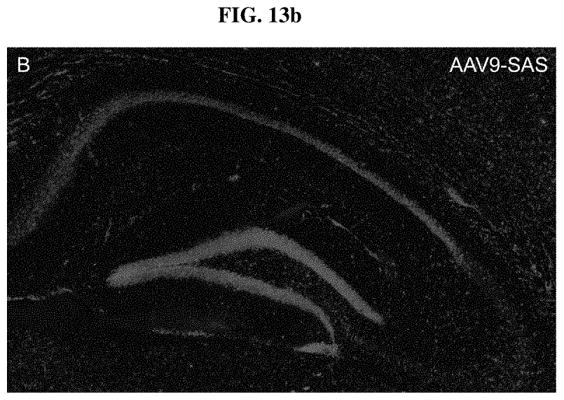

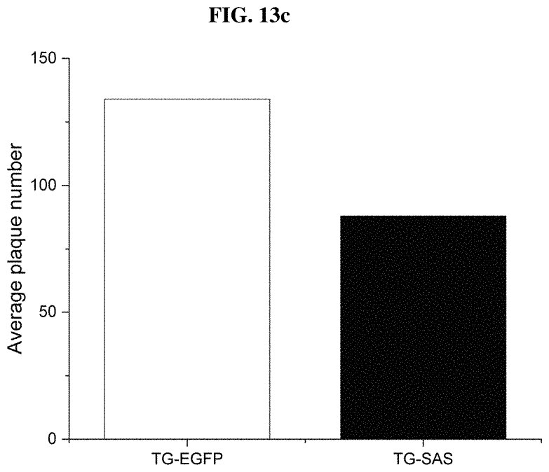

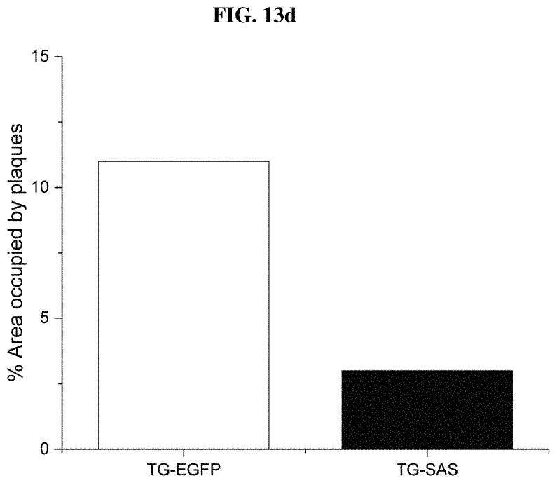

After extracting the brains of the mice used in Experimental Example 6, amyloid plaques were observed through immunohistochemical analysis, and the results are shown in FIGS. 13a to 13d.

As shown in FIGS. 13a and 13b, it was confirmed that plaques stained red with Aβ antibody were accumulated in the hippocampus, which controls memory ability, in the brain of the mice of the TG-EGFP group, and these plaques were reduced in the TG-SAS group. Additionally, through the quantitative analysis of FIGS. 13c and 13d, it was confirmed that the TG-SAS group showed a decrease in both the number and area of plaques compared to the TG-EGFP group. Therefore, it was confirmed that the expression of SAS through AAV9 showed a therapeutic effect on reducing plaques accumulated in the brain of 5XFAD mice.

1. A fusion protein of Formula 1 below:
(X)ₘ-(L1)ₙ-A-(L2)ₒ-(Y)ₚ-(Z)_{q} [Formula 1]
wherein in Formula 1 above,
X is a signal sequence;
L1 is peptide linker 1;
A is a nuclear inclusion a (NIa) protease or a fragment thereof, or a variant thereof;
L2 is peptide linker 2;
Y is a transmembrane domain;
Z is an intracellular domain; and
m, n, o, p, and q are each 0 or 1.

2. The fusion protein of claim 1,
wherein the signal sequence is derived from β-secretase.

3. The fusion protein of claim 1,
wherein the signal sequence is a polypeptide having the amino acid sequence of SEQ ID NO: 3.

4. The fusion protein of claim 1,
wherein the peptide linker 1 is a peptide consisting of 3 to 24 amino acids.

5. The fusion protein of claim 1,
wherein the peptide linker 1 is derived from β-secretase.

6. The fusion protein of claim 1,
wherein the peptide linker 1 is a polypeptide represented by amino acids of SEQ ID NOS: 5 to 11.

7. The fusion protein of claim 1,
wherein the NIa protease is derived from the family Potyviridae.

8. The fusion protein of claim 1,
wherein the NIa protease has an activity to degrade amyloid β.

9. The fusion protein of claim 1,
wherein the NIa protease is a polypeptide represented by the amino acid sequence of SEQ ID NO: 12.

10. The fusion protein of claim 1,
wherein the NIa protease variant is one in which one of the N-glycosylation sites of the NIa protease is substituted with another amino acid.

11. The fusion protein of claim 10,
wherein the another amino acid is any one selected from the group consisting of arginine (Arg), histidine (His), lysine (Lys), aspartic acid (Asp), glutamic acid (Glu), serine (Ser), threonine (Thr), glutamine (Gln), tyrosine (Tyr), alanine (Ala), isoleucine (Be), leucine (Leu), valine (Val), phenylalanine (Phe), methionine (Met), tryptophan (Trp), glycine (Gly), proline (Pro), and cysteine (Cys).

12. The fusion protein of claim 10,
wherein the N-glycosylation site is N70, N115, or N253 in the amino acid sequence of SEQ ID NO: 31.

13. The fusion protein of claim 1,
wherein the transmembrane domain is derived from any one selected from the group consisting of BACE, ELAVL3, NRGN, REEP2, GAD1, PCDHA1, GFAP, S100B, FAM19A1, AQP4, and CLEC2L.

14. The fusion protein of claim 1,
wherein the transmembrane domain is a polypeptide represented by the amino acid sequence of SEQ ID NO: 29.

15. The fusion protein of claim 1,
wherein the intracellular domain is derived from β-secretase.

16. The fusion protein of claim 1,
wherein the intracellular domain is a polypeptide having the amino acid sequence of SEQ ID NO: 30.

17. A polynucleotide encoding the fusion protein according to claim 1.

18. The polynucleotide of claim 17,
wherein the polynucleotide is a polynucleotide having the nucleotide sequence of SEQ ID NO: 42.

19. An expression vector comprising the polynucleotide of claim 17.

20. A recombinant virus comprising the polynucleotide of claim 17.

21. A host cell transfected with the expression vector of claim 19.

22. The host cell of claim 21,
wherein the host cell is any one selected from the group consisting of E. coli, CHO cells, and HEK293 cells.

23. A method of treating diseases and conditions characterized by excessive accumulation of amyloid β comprising:
administering a therapeutically effective amount of a pharmaceutical composition comprising a polynucleotide encoding for a fusion protein according to Formula 1:
(X)ₘ-(L1)ₙ-A-(L2)ₒ-(Y)ₚ-(Z)_{q} (1)
wherein,
X is a signal sequence;
L1 is peptide linker 1;
A is a nuclear inclusion a (NIa) protease, or a fragment or a variant thereof;
L2 is peptide linker 2;
Y is a transmembrane domain; and
Z is an intracellular domain,
wherein m, n, o, p, and q are each 0 or 1.

24. The method of claim 23,
wherein the disease or condition is selected from the group consisting of Alzheimer's disease, mild cognitive impairment (MCI), mild-to-moderate cognitive impairment, vascular dementia, cerebral amyloid angiopathy, hereditary cerebral hemorrhage, senile dementia, Down syndrome, inclusion body myositis, age-related macular degeneration, and conditions associated with Alzheimer's disease.

25. The method according to claim 23, wherein:
the polynucleotide is contained within a recombinant virus.

26. The method according to claim 25, further comprising:
selecting the virus from the group consisting of retroviruses, lentiviruses, adenoviruses, adeno-associated viruses (AAV), herpesviruses, poxviruses, baculoviruses, papillomaviruses, vaccinia viruses, parvoviruses, and mixtures thereof.

27. The method according to claim 23, further comprising:
initiating administration of the therapeutically effective amount of the pharmaceutical composition to a susceptible individual before detecting any symptoms associated with the disease or condition.
